(19) United States
(12) Patent Application Publication
(10) Pub. No.: US 2025/0331949 A1
(43) Pub. Date: Oct. 30, 2025

(54) MEDICAL IMAGING DEVICE

(71) Applicant: Carl Zeiss Meditec AG, Jena (DE)

(72) Inventors: Marcel Walch, Ulm (DE); David Dobbelstein, Ulm (DE); Thomas Lindemeier, Dornstadt (DE); Steffen Urban, Jena (DE); Daniel Werdehausen, Herbrechtingen (DE)

(73) Assignee: Carl Zeiss Meditec AG, Jena (DE)

(21) Appl. No.: 19/186,656

(22) Filed: Apr. 23, 2025

(30) Foreign Application Priority Data
Apr. 24, 2024 (DE) 10 2024 111 596.3

(51) Int. Cl. A61B 90/00; G16H 30/40

(52) U.S. Cl. CPC A61B 90/361; G16H 30/40; A61B 2090/365

(57) ABSTRACT

A medical imaging device comprises an image recording device for stereoscopic image recording. The medical imaging device further comprises an internal environmental sensor which is mounted on the medical imaging device and is configured to capture environmental sensor data relating to an environment of the medical imaging device, and a data processing device which is connected to the internal environmental sensor and is configured to receive the environmental sensor data captured by the internal environmental sensor and to create a three-dimensional environmental model of the environment of the medical imaging device based on the received environmental sensor data.

### CROSS REFERENCE TO RELATED APPLICATIONS

This application claims priority to German patent application 10 2024 111 596.3 filed on Apr. 24, 2024, which is hereby incorporated by reference in its entirety.

### TECHNICAL FIELD

The present disclosure relates to a medical imaging device which comprises an image recording device for stereoscopic image recording and is configured to create a three-dimensional environmental model of the environment of the medical imaging device based on environmental sensor data captured by an internal environmental sensor.

### BACKGROUND

U.S. Pat. No. 11,769,302 B2 concerns creating a virtual representation of an operating theater. The virtual representation is created on the basis of robot information and a scan of the operating theater with depth cameras. One of the depth cameras is integrated in a portable electronic apparatus operated by a local user in the operating theater. The virtual representation of the operating theater is transmitted to a virtual reality headset together with three-dimensional point cloud data. A virtual reality environment is displayed on a display of the virtual reality headset operated by a remote user. A virtual representation of the remote user is displayed in augmented reality on a display of the portable electronic apparatus.

U.S. Pat. No. 11,756,672 B 2 relates to a surgical procedure performed with a surgical robot system. The procedure is captured by depth cameras that generate 3D point cloud data. The data from the robot system that are associated with the surgical robot system are recorded. Object recognition is performed using the image data generated by one or more depth cameras in order to recognize objects, including surgical apparatuses and people, in the operating theater. The surgical procedure is digitized by storing the 3D point cloud data relating to the unrecognized objects, a position and orientation associated with the recognized objects, and the robot system data.

US 2020/315734 A 1 relates to a method for using a surgical visualization system during a surgical procedure. The method comprises the steps of capturing patient reference data, loading the patient reference data and features into the computer, and capturing live data from the operating theater during the surgical procedure, where the live data comprise a three-dimensional live model of the patient. The method continues with the steps of registering the patient reference data and live data and displaying a real-time overlay of selected registered patient reference data on the patient through a headset worn by the surgeon.

US 2021/335483 A 1 relates to a surgical visualization theater comprising the following: an augmented reality headset, a digital viewing window mounted on a cobot arm provided for this purpose, a monitor mounted on a cobot arm provided for this purpose, a camera subsystem mounted on a cobot arm provided for this purpose, and a frame with cobot arms that have intelligence and command and control functions for the system and the visualization methods, wherein the cobot arm for the digital viewing window, the cobot arm for the monitor and the cobot arm for the camera are mounted on the frame and the headset is connected thereto.

In microsurgery, stereoscopic visualization systems, such as the conventional surgical microscope, are indispensable, since surgeons rely on the greatly magnified stereoscopic view in order to perform their surgical tasks.

In order to obtain a well-oriented visual perspective on the surgical area during the operation, precise settings, such as of the positioning, light, zoom and focus properties, of the surgical microscope are required.

These settings require frequent manual interventions by the surgeon in conventional systems and can therefore interfere with a course of the operation.

Since modern surgical microscopes, especially their microscope stands, are robot-controlled and the microscope head is equipped with digital (stereo) cameras (and possibly also other sensors such as IM Us, proximity sensors, etc.), it would be advantageous if the surgical microscope could perform movements and/or setting adjustments in an automated manner, e.g. in a partially automated up to fully autonomous manner.

US 2022/0096197 relates to an augmented reality headset (AR headset) that provides the wearer with spatial, system-related and temporal context information relating to a surgical robot system in order to assist the wearer with configuring, operating, or troubleshooting the surgical robot system before, during or after an operation. Spatial context information can be rendered in order to display spatially fixed 3D-generated virtual models of the robot arms, instruments, the bed, and other components of the surgical robot system that correspond to the actual position or orientation of the surgical robot system in the coordinate system of the AR headset. The AR headset can communicate with the surgical robot system in order to obtain real-time state information about the components of the surgical robot system. The AR headset can use the real-time state information to display context-dependent user interface information such as tips, suggestions, visual or audible cues for manoeuvring the robot arms and the table to their target positions and orientations, or for troubleshooting purposes.

### SUMMARY

A medical imaging device is provided. The medical imaging device comprises an image recording device for stereoscopic image recording, an internal environmental sensor which is mounted on the medical imaging device and is configured to capture environmental sensor data relating to an environment of the medical imaging device, and a data processing device which is connected to the internal environmental sensor and is configured to receive the environmental sensor data captured by the internal environmental sensor and to create a three-dimensional environmental model of the environment of the medical imaging device based on the received environmental sensor data.

### BRIEF DESCRIPTION OF THE DRAWINGS

An optional embodiment is described below with reference to FIGS. 1 to 10.

FIG. 1 perspectively and schematically shows a medical imaging device according to the disclosure,

FIG. 2 schematically shows the imaging device according to the disclosure from FIG. 1 in an exemplary situation in an operating room,

FIG. 3 schematically shows a structure of a data processing device, which is part of the medical imaging device according to the disclosure from FIG. 1,

FIG. 4 schematically shows a flowchart of a method according to the disclosure,

FIG. 5 shows a schematic diagram in which a spatial relationship of the medical imaging device, the microscope, the internal environmental sensor and the external environmental sensor is shown,

FIG. 6 schematically shows a flowchart of a first computer-implemented method of the first exemplary embodiment for determining the first transformation rule,

FIG. 7 schematically shows a flowchart of a second computer-implemented method of the first exemplary embodiment for determining the first transformation rule,

FIG. 8 schematically shows a flowchart of a third computer-implemented method of the first exemplary embodiment for determining the first transformation rule,

FIG. 9 schematically shows a flowchart of a computer-implemented method of a second exemplary embodiment for determining the first transformation rule, and

FIG. 10 schematically shows a flowchart of a computer-implemented method of a third exemplary embodiment for determining the first transformation rule.

### DESCRIPTION OF EXEMPLARY EMBODIMENTS

In the following, details are set forth to provide a more thorough explanation of the disclosure. However, it will be apparent to those skilled in the art that these implementations may be practiced without these specific details. In other instances, well-known structures and devices are shown in block diagram form or in a schematic view rather than in detail in order to avoid obscuring the disclosure. In addition, features described hereinafter may be combined with each other, even if described with respect to different figures, unless specifically noted otherwise.

Equivalent or like elements or elements with equivalent or like functionality are denoted in the following description with equivalent or like reference numerals. As the same or functionally equivalent elements are given the equivalent or like reference numbers in the figures, a repeated description for elements provided with the equivalent or like reference numbers may be omitted. Hence, descriptions provided for elements having the equivalent or like reference numbers are mutually exchangeable.

Directional terminology, such as “top,” “bottom,” “below,” “above,” “front,” “behind,” “back,” “leading,” “trailing,” etc., may be used with reference to the orientation of the figures being described. Because parts of the disclosure, described herein, can be positioned in a number of different orientations, the directional terminology is used for purposes of illustration and is in no way limiting. It is to be understood that other implementations may be utilized, and structural or logical changes may be made without departing from the scope defined by the claims. The following detailed description, therefore, is not to be taken in a limiting sense.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In implementations described herein or shown in the drawings, any direct electrical connection or coupling, e.g., any connection or coupling without additional intervening elements, may also be implemented by an indirect connection or coupling, e.g., a connection or coupling with one or more additional intervening elements, or vice versa, as long as the general purpose of the connection or coupling, for example, to transmit a certain kind of signal or to transmit a certain kind of information, is essentially maintained. Features from different implementations may be combined to form further implementations. For example, variations or modifications described with respect to one of the implementations may also be applicable to other implementations unless noted to the contrary.

The terms “substantially” and “approximately” may be used herein to account for small manufacturing tolerances (e.g., within 5%) that are deemed acceptable in the industry without departing from the aspects of the implementations described herein. For example, a resistor with an approximate resistance value may practically have a resistance within 5% of that approximate resistance value.

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

A medical imaging device is provided, which comprises an image recording device for stereoscopic image recording. The medical imaging device comprises an internal environmental sensor which is mounted on the medical imaging device and is configured to capture environmental sensor data relating to an environment of the medical imaging device. The medical imaging device comprises a data processing device connected to the internal environmental sensor. The data processing device is configured to receive the environmental sensor data captured by the internal environmental sensor and to create a three-dimensional environmental model of the environment of the medical imaging device based on the received environmental sensor data.

The medical imaging device can be understood as meaning, for example, a surgical microscope. The surgical microscope can be used, for example, in a minimally invasive surgical procedure and in microsurgery.

The environmental sensor may be a monocular and/or stereoscopic RGB camera, an RGBD sensor, an infrared or near infrared sensor, a time-of-flight sensor and/or a sensor that uses structured light. A plurality of the environmental sensors may be provided.

The environment of the medical imaging device can be understood as meaning, for example, an operating room or a part of an operating room in which the medical imaging device is located. An operating room can be understood as meaning a room or an area which is set up for the surgical treatment of the living being both structurally and by way of the medical apparatuses present therein. The operating room or operating theater is often a special room in a hospital or doctor's office in which surgical procedures, the operations, are performed. However, the operating room can be broadly understood in the present case, and so can be any room in which the living being can be treated. The treatment can be an operation, but can also be any other type of medical treatment, such as a diagnostic examination using an imaging method.

The environment can optionally assume different dimensions depending on a configuration of the sensors. For example, it may be an environment of the surgical area. It may also be, for example, a volume that includes a patient and/or their immediate environment, such as a person on the operating table and/or other objects in this area (e.g. sterile zone). It can also be a complete (operating) room, for example. The environment can have a volume of 0.5 m*0.5 m*0.3 m (0.3 m=variable working distance of the microscope) up to several cubic meters. If the volume covers substantially the entire operating room, this may have the following external dimensions, for example:
Height: 2.0 m to 5.0 m, optionally greater than or equal to 3.0 m (since such a clear room height, i.e. the top edge of the finished floor to the bottom edge of the suspended ceiling, of an operating room should be provided for the apparatuses and/or persons required to perform the treatment)
Size: 10 m² to 60 m² or larger (so that there is sufficient space for the surgical personnel and/or the apparatuses required to perform the treatment, depending on the treatments to be performed therein).

In a specific example which does not restrict the disclosure, the environment can be a cuboid volume with a base area of 8 m by 8 m and a height of 3 m.

The image recording device for stereoscopic image recording can also be referred to as a stereoscopic image sensor. It is conceivable that the stereoscopic image sensor can be used to record or generate a three-dimensional image of an operating region. The stereoscopic image sensor can be in the form of a microscope. A stereoscopic image sensor can be understood as meaning a sensor which is configured to record at least two images of the same part of the surrounding area from (slightly) different perspectives. The microscope may have a separate beam path for each eye of an observer. The stereoscopic image sensor can be used to record two images from (slightly) different perspectives, resulting in a stereo or 3D effect for the viewer.

The three-dimensional environmental model can be created continuously, optionally in real time. The creation of the three-dimensional environmental model can be considered to be a computer-implemented method, i.e. one, multiple or all steps of the method can be carried out at least partially by a computer or a data processing device. Furthermore, the disclosure relates to a computer program comprising instructions which, when the program is executed by a computer, cause the computer to carry out the method. A program code of the computer program can be present in any desired code, optionally in a code suitable for controllers of a medical imaging device. Furthermore, the disclosure relates to a computer-readable medium comprising instructions which, when the instructions are executed by a computer, cause the computer to carry out the method. That is to say that a computer-readable medium comprising a computer program defined above can be provided. The computer-readable medium can be any desired digital data storage apparatus, such as for example a USB stick, a hard disk, a CD-ROM, an SD card or an SSD card (or SSD drive/SSD hard disk). The computer program need not necessarily be stored on such a computer-readable storage medium in order to be made available to the computer, but rather can also be obtained externally via the Internet or in some other way. In other words, the computer-readable medium may be a data signal comprising instructions which, when the instructions are executed by the computer, cause the computer to carry out at least one of the methods described above. The applicant reserves the right to file a divisional application directed to the method, the computer program and/or the computer-readable medium.

The 3D environmental model can be understood as meaning a digital and three-dimensional representation of the environment, optionally of an operating room or operating theater, with static and/or dynamic objects located therein.

The medical imaging device may include a base on which a stand is movably mounted. The data processing device can be arranged in and/or on the base. However, it is also conceivable for the data processing device to be arranged in an at least partially remote or distant manner from the medical imaging device and to be able to communicate or exchange data, wirelessly and/or in a wired manner, with units arranged on and/or in the medical imaging device. The base can be moved manually and/or automatically. The stand can be moved manually and/or automatically relative to the base. The movement of the stand relative to the base can be carried out translationally and/or rotationally. The stand can be moved via one or more servomotors which are controlled by the data processing device based on the generated 3D environmental model and/or a user input. The image recording device for stereoscopic image recording may be mounted on one end of the stand opposite the end of the stand mounted on or fastened to the base. The environmental sensor(s) may be mounted on the base and/or the stand. It is conceivable for at least one of the environmental sensors to be mounted on that end of the stand on which the image recording device for stereoscopic image recording is also mounted. A field of view of the image recording device for stereoscopic image recording and a field of view of at least one of the environmental sensors, optionally of the environmental sensor mounted on that end of the stand on which the image recording device for stereoscopic image recording is also mounted, may overlap. Optionally, the field of view of the image recording device for stereoscopic image recording may be arranged, further optionally completely, within the field of view of the at least one environmental sensor.

The medical imaging device described above offers a number of advantages which are described below.

A description is given of a device for a robotic stereoscopic visualization system which uses 3D environmental perception that makes it possible to create a 3D model of an operating environment. In turn, the 3D model can be used to derive properties that can be used to warn of, recommend and/or automatically initiate system adaptations of the visualization system.

An operator of the visualization system can be assisted in this way, since increasing automation of the operation of the visualization system means that fewer manual interventions for operation are required and interruptions in a course of the operation can thus be reduced. This can result in an increase in efficiency and the patient outcome.

The proposed visualization system uses an inside-out tracking approach, i.e. the (environmental) sensor system is integrated in or mounted on the visualization system itself and can thus provide egocentric data from a viewing angle that is at the center of the surgical procedure. This is an advantage over external sensors, such as systems mounted on the ceiling, because the environmental sensor mounted on the device has an unobstructed view of the situs, the patient, and the surgeon and their hands.

Possible developments of the device described above are explained in detail below.

The data processing device may be configured to be connected to the image recording device in order to receive stereoscopic image data from the image recording device, and to fuse the received stereoscopic image data with the environmental sensor data received from the internal environmental sensor for the purpose of generating the three-dimensional environmental model.

This makes it possible to additionally include the stereoscopic imaging in the generation of the three-dimensional environmental model, which can be used, for example, to reconstruct the situs and/or to track tools or surgical equipment. Situs can be understood as meaning a region to be operated on and optionally an area surrounding it, e.g. open skull, back with internal organs, etc.

The data processing device may be configured to be connected to an environmental sensor external to the medical imaging device in order to receive further environmental sensor data from the external environmental sensor, and to fuse the further received environmental sensor data with the environmental sensor data received from the internal environmental sensor and/or the stereoscopic image data received from the image recording device for the purpose of generating the three-dimensional environmental model.

This means that one or more environmental sensors installed and/or mounted in the (optionally operating theater) room can also be used to generate the 3D environmental model of the (optionally operating theater) environment. It is also possible to use additional environmental sensors, which are integrated in head-mounted visualization systems (head-mounted device, HMD), to generate the 3D environmental model of the (optionally operating theater) environment.

In other words, in order to create the 3D environmental model, the microscope must perceive its surrounding area and classify the objects therein and/or their surfaces, such as medical personnel, other apparatuses, the patient's position, etc. Since the microscope or the image recording device itself has only a very limited field of view of the surrounding area (i.e. the sensor in the microscope head is directed downward toward the patient and the floor), in addition to the microscopic stereo image of the situs, further sensors can be included in the creation of a dynamic map of the operating theater scene or the 3D environmental model. For example, these may be cameras on other screens, on the ceiling, and/or head-mounted AR/MR/VR systems. It is therefore possible to provide a sensor data fusion in which sensor data from sensors not mounted on the device are fused with sensor data from the sensors mounted on the device. It is conceivable that the environmental sensor data from the external and/or internal environmental sensor and/or the stereoscopic image data can be dynamically used, i.e. added or removed, when generating the environmental model.

All sensors or apparatuses may be or may have been registered in a common reference map, and so they interact, move and avoid collisions, and/or the current relationships and/or positions of objects and/or persons in the room can be taken into account, during operation of the medical imaging device. A three-dimensional, semantically enriched map of the environment can be created, i.e. the 3D environmental model. This map can be represented explicitly (for example as a point cloud, a grid, voxels and labels for points or triangles, the position, orientation, and class of objects in the room) and/or implicitly (i.e. the information can be coded in a neural network, for example). In other words, the surface of objects, persons and/or the operating room can be represented as a point cloud using a multiplicity of data points in a 3D coordinate system in order to obtain the map. Additionally or alternatively, it is possible to affect a representation as a polygon mesh, the faces of which reproduce the surface of the aforementioned objects and the surrounding area, in order to obtain the map. Additionally or alternatively, the map can also be represented less objectively, and so it can also consist of basic geometric shapes that enclose the objects, persons etc. (e.g. 3D Bounding Boxes). Additionally or alternatively, it is conceivable that 3D models of the apparatuses or persons are displayed at the respective point in the map. The different data structures can be supplemented with semantic information, such as the class (i.e. which apparatus) and/or the identity (i.e. which person) to which this data item belongs.

It is conceivable that a first coordinate system is defined for the image recording device and/or the internal environmental sensor, the coordinates of which indicate a position of a point in the environment of the medical imaging device relative to the image recording device and/or the internal environmental sensor. A second coordinate system may be defined for the external environmental sensor, the coordinates of which indicate a position of a point in the environment of the medical imaging device relative to the external environmental sensor. The fusion of the further environmental sensor data with the stereoscopic image data and/or the environmental sensor data may comprise determining a first transformation rule, by means of which coordinates of the second coordinate system can be converted into coordinates of the first coordinate system, and/or vice versa.

This means that each sensor can have its own local coordinate system, wherein the situation, i.e. the position and/or orientation, of objects and/or surfaces, which are contained in the respective sensor data, can be determined or detected by the sensor in the respective local coordinate system.

A local coordinate system can be understood as meaning a coordinate system whose origin and orientation are fixed relative to the respective sensor. This means that, if the sensor changes its situation in space, the situation of the local coordinate system also changes with it.

So that the information regarding the situation of the objects and/or surface can now be combined in a single 3D environmental model, a so-called transformation rule is provided and allows the positions and/or orientations from the local coordinate systems to be converted into a single target coordinate system. This target coordinate system may be, for example, the local coordinate system of the image recording device and/or the internal environmental sensor. Additionally or alternatively, the target coordinate system can be fixed on the base. However, the target coordinate system can also be a global coordinate system.

A global coordinate system can be understood as meaning a coordinate system whose origin and orientation are fixed in space. This means that the situation of the global coordinate system remains the same regardless of whether the situation of an object in space changes.

The positions and/or orientations from the local coordinate systems can be converted or combined directly, i.e. the respective coordinates are converted directly from the local coordinate system into the target coordinate system, or indirectly, i.e. a relative situation of the sensors with respect to each other is first determined and then, based on the relative situation of the sensors with respect to each other, the geometric relationship between the sensor data captured by the sensors is modeled.

The conversion of the positions and/or orientations from the local coordinate systems into a single target coordinate system is challenging in the present application, among other things due to the size differences between the fields of view of the individual sensors, the position changes of the sensors with respect to each other and in space, the different sampling rates and map representations of the sensors as well as the constantly changing scale due to zoom/focal length changes.

In detail, a stereo microscope can have a field of view in the scene of 0.10 m×0.10 m with a camera resolution of 4000×4000 pixels, for example with an object distance of 0.3 m, resulting in 400 samples/cm. For example, the external environmental sensor can be an RGBD camera that has a field of view in the scene of 0.74 m×0.74 m with a resolution of 544×544 pixels with an object distance of 0.5 m, resulting in approximately 7 samples/cm.

This large difference in the sampling rates makes it challenging to determine the first transformation rule. In addition, not only the sampling rates, but also the accuracy and thus the surface noise of the individual sensor data types are very different, which leads to challenges in feature extraction, matching and surface orientation. In addition, in the present application, the overlap of the fields of view of the individual sensors can be extremely small, e.g. only 0.07% (for example, with a volume of 0.125 m³ scanned by the microscope (0.5 m×0.5 m×0.5 m cube) compared to 192 m³ (8 m×8 m×3 m operating theater). Various possible ways of performing a sensor data fusion efficiently and accurately despite these challenges are described below.

It is conceivable that a third coordinate system is defined for the medical imaging device, the coordinates of which indicate a position of a point in the environment of the medical imaging device relative to the medical imaging device. A second transformation rule can be determined, by means of which coordinates of the first coordinate system can be converted into coordinates of the third coordinate system, and/or vice versa. The determination of the first transformation rule may comprise determining a third transformation rule, by means of which coordinates of the second coordinate system can be converted into coordinates of the third coordinate system, and/or vice versa. The first transformation rule can be determined based on the second and third transformation rules.

That is to say, it is proposed to determine the first transformation rule using an intermediate step. In the intermediate step, a respective transformation rule is determined that allows the coordinates from the local coordinate system of the external environmental sensor or the internal environmental sensor and/or the image recording device to be converted into a common coordinate system. A combination of these two transformation rules determined in the intermediate step then results, for example by multiplication, in the transformation rule which describes the relationship between the two local coordinate systems of the external environmental sensor or the internal environmental sensor and/or the image recording device. The common coordinate system may be a local coordinate system of the medical imaging device, optionally of the base of the medical imaging device. The advantage of the intermediate step is that the sensor data with the highly different properties described above do not have to be directly compared with each other, and so the resulting challenges described above can be circumvented.

The data processing device may be configured to determine the second transformation rule based on a current position and/or orientation of the image recording device and/or the internal environmental sensor, optionally represented by a current position and/or orientation of the microscope head, in space.

In other words, it is conceivable that the second transformation rule, i.e. that transformation rule by means of which the coordinates of the local coordinate system of the internal environmental sensor and/or the image recording device can be converted into the coordinates of the local coordinate system of the medical imaging device, is determined based on a situation of the internal environmental sensor and/or the image recording device relative to the medical imaging device, optionally its base. The current situation of the internal environmental sensor and/or the image recording device can be determined, for example, based on a current calibration of the medical imaging device and/or based on the environmental sensor data from the external environmental sensor, when the medical imaging device is (sufficiently) represented in the environmental sensor data from the external environmental sensor.

It is conceivable that the medical imaging device is represented as a first point cloud in the further environmental sensor data. The determination of the third transformation rule can comprise creating a 3D image of the medical imaging device based on a current position and/or orientation of the image recording device in space, deriving a second point cloud based on the 3D image of the medical imaging device, which optionally has the same density as the point cloud representing the medical imaging device in the further environmental sensor data, and determining the third transformation rule by comparing the first point cloud with the second point cloud.

In other words, a 3D-3D registration is proposed. A point cloud can be derived from the 3D image by sampling the 3D image. In addition, it is possible to derive normal directions, colors, and/or other 3D features that are useful for the comparison. This point cloud can be sampled with a similar density to the point cloud of the external environmental sensor. Optionally, the point cloud of the external environmental sensor can be pre-processed in order to remove unwanted objects, such as persons, walls, floors, etc., and thus facilitate the comparison. Finally, the point clouds of the external environmental sensor and the point cloud derived from the 3D image can be compared. In a final step, the final transformation can be calculated. This can be carried out for multiple registrations in a video stream, and/or an optimization can be performed using the least squares method in order to compensate for errors.

The 3D image can be a rigged/textured digital twin (3D/CAD model) of the medical imaging device. “Rigged” means that the modeled joints of the robot or the medical imaging device can move. The forward and reverse kinematics of the robot are known. This information can be used to mirror the exact orientation of the real robot in the digital twin (3D model). In a surgical environment, parts of the medical imaging device may be covered with a drape, yet the solutions proposed herein work even if only the uncovered parts of the medical imaging device are used instead of the entire robot model.

It is therefore proposed to derive a pose of a target point cloud (i.e. the transformation from the medical imaging device to the image recording device and/or the internal environmental sensor) in the local coordinate system of the medical imaging device from the calibration and/or the geometric 3D model of the robot of the medical imaging device. Then, when the transformation from the source coordinate system to the robot coordinate system (i.e. the transformation from the external environmental sensor to the medical imaging device) is determined, the source or the external environmental sensor can be aligned with the target coordinate system of the image recording device and/or the internal environmental sensor by using the geometric 3D model of the robot as an intermediate step.

It is conceivable that the medical imaging device is represented as a first 2D image in the further environmental sensor data. The determination of the third transformation rule may comprise obtaining a 3D image of the medical imaging device, deriving at least one second 2D image of the medical imaging device from its 3D image in order to determine a position of the external environmental sensor in the third coordinate system, from which the first 2D image of the medical imaging device was recorded, and determining the third transformation rule based on the determined position of the external environmental sensor in the third coordinate system, from which the first 2D image of the medical imaging device was recorded.

In other words, as an alternative to the 3D-3D registration, a 2D-3D registration is proposed. Synthetic views of the 3D image can be rendered with a similar camera projection to that of the source cameras. Instead of rendering only RGB images, depth and normal images of the medical imaging device can also be rendered. In addition, a-priori information can be used to restrict the virtual poses from which the views are rendered (e.g., if the external environmental sensor is part of a head-mounted device, a height of the external environmental sensor may result from a height of the person wearing the device, and its orientation may result from a typical viewing direction of this person).

The determination of the position of the external environmental sensor in the third coordinate system, from which the first 2D image of the medical imaging device was recorded, may comprise deriving a plurality of the second 2D images of the medical imaging device from its 3D image, as seen from different positions in the third coordinate system. The determination of the position of the external environmental sensor in the third coordinate system may comprise extracting a predetermined set of features, optionally comprising 2D features and/or image gradients, from each of the first and second 2D images. The determination of the position of the external environmental sensor in the third coordinate system may comprise selecting one of the second 2D images based on a comparison of the extracted sets of features of the second 2D images with the extracted set of features of the first 2D image. The determination of the position of the external environmental sensor in the third coordinate system may comprise adopting the position in the third coordinate system, from which the selected second 2D image was derived, as the position of the external environmental sensor in the third coordinate system, from which the first 2D image of the medical imaging device was recorded.

This means that, in one possible implementation of the 2D-3D registration, 2D features can be extracted from the rendered 2D views. The same features can also be extracted from the sensor data from the external environmental sensor. In a next step, all rendered images or 2D views can be used to determine the one that comes closest to the 2D view recorded by the external environmental sensor. This can be carried out either by fully matching all features to each other or by means of a search tree/indexing method, such as the nearest neighbor search. Finally, it is possible to estimate the pose of the medical imaging device in the 2D view recorded by the external environmental sensor. For this purpose, 2D-to-2D correspondences can be established first, i.e. the best matching 2D features between the features extracted from the respective 2D views can be determined. Since the rendered views can also contain depth information, the 2D features can now be converted into 3D points, thus obtaining 2D-3D correspondences. Now, any Perspective-n-Point (PnP) solver can be used to determine the position of the external environmental sensor in the third coordinate system.

In a possible further implementation of the 2D-3D registration, lines or contours can be used to estimate the pose or the transformation matrix from the coordinate system of the medical imaging device into the coordinate system of the external environmental sensor. For this purpose, line or edge or gradient features can be extracted from all views rendered by the 3D model of the medical imaging device. This can take place on the 2D images rendered as RGB images alone or can be further improved by also extracting lines or edges or gradients from normal and/or depth images. This is especially useful for textureless object regions. Line/edge/gradient features can then also be extracted from the 2D views of the external environmental sensor. In a next step, all rendered images can be used to determine the image that comes closest to the current image captured by the external environmental sensor. This can take place with the aid of visual location recognition and/or by calculating a similarity metric for the gradient images, e.g. cosine similarity. Finally, the pose of the image captured by the external environmental sensor can be estimated with the aid of PnP, Perspective-n-Line (PnL) or edge-based iterative pose optimization in order to compare the gradient image from the external environmental sensor with the selected rendered gradient image. In other words, gradients or edges can be extracted from the real camera stream or the environmental sensor data recorded by the external environmental sensor. Gradients or edges can also be extracted from the rendered images of the virtual object (that is to say, the 3D model of the medical imaging device). The gradients or edges extracted from the camera stream can be compared with the gradients or edges from the rendered images. Images of the virtual object can be rendered from different poses until the extracted gradients or edges match the edges extracted from the camera stream during the comparison. If the gradients or edges of both images match, the pose of the real camera relative to the virtual object is known due to the given pose of the virtual camera.

A normal image or a normal map can be understood as meaning a three-channel image, with the channels each corresponding to the X, Y and Z coordinates of the surface normals. For depth images, each pixel can store the depth from the pinhole or the image plane to the object. If both images are combined and edges or gradients are extracted, a quasi-complete contour image of an object can be obtained.

It is conceivable that the medical imaging device is represented as a first 2D image in the further environmental sensor data. The determination of the third transformation rule may comprise obtaining a 3D image of the medical imaging device and deriving a plurality of the second 2D images of the medical imaging device from its 3D image, as seen from different positions in the third coordinate system. The determination of the third transformation rule may comprise determining an implicit scene representation, for example training a neural radiance field, based on the plurality of second 2D images and the respective position in the third coordinate system, from which the respective second 2D image was derived. The determination of the third transformation rule may comprise generating further 2D images of the medical imaging device by means of the determined implicit scene representation using various transformation rules, wherein the further 2D images are generated iteratively until an image reconstruction loss determined based on a last generated further 2D image and the first 2D image falls below a predetermined limit value. The determination of the third transformation rule may comprise adopting the transformation rule, according to which the last generated further 2D image was generated, as the third transformation rule.

In other words, in a possible further implementation of the 2D-3D registration, it is possible to implicitly calculate the transformation rule from the external environmental sensor to the imaging medical device. First, a 3D CAD model of the imaging medical device can be rendered from a plurality of views. An implicit scene representation NF can then be generated, for example as a neural radiance field or a Gaussian splatting model. The advantage of this is that further 2D views can be synthesized from this implicit scene representation in a completely differentiable way. Therefore, it is possible to create a loss function that optimizes an image reconstruction loss, that is to say minimizes the pixel intensity differences:

$${min}_{{\hat{T}}_{source}^{robot}} {\sum }_{j}^{WH} ‖ {I}_{syn} ( {\hat{T}}_{source}^{robot} \vert NF ) - {I}_{s} ‖$$

W and H are the width and height of the image.

$${I}_{syn} ( {{\hat{T}}^{′}}_{source}^{robot} \vert NF )$$

is a synthesized image from the pose

$${{\hat{T}}^{′}}_{source}^{robot}$$

taking into account the trained neutral radiance field. The loss is fully differentiable; therefore, the pose can be optimized using the gradient descent in order to find the optimal pose at which the source image was recorded relative to the robot.

Alternatively, the determination of the first transformation rule may comprise converting coordinates, which indicate a situation of the stereoscopic image data and/or the environmental sensor data in the first coordinate system and a situation of the further environmental sensor data in the second coordinate system, into a third coordinate system, the vertical axis of which runs parallel to the effective direction of the gravitational force, in order to obtain a situation of the stereoscopic image data and/or the environmental sensor data and the further environmental sensor data in the third coordinate system. The determination of the first transformation rule may comprise determining the first transformation rule using a predetermined cross-source point cloud registration method based on the situation of the stereoscopic image data and/or the environmental sensor data and the further environmental sensor data in the third coordinate system.

That is to say, unlike the solutions described above, in which the medical imaging device is used as a common object to bypass the direct alignment of target with source sensor data and indirectly align it via the 3D model of the robot, the direct alignment of target with source sensor data can now be implemented in this solution. One of the challenges in the direct alignment of target with source sensor data can be the different sampling density and the small overlap of the two sets of sensor data, optionally each available as a point cloud. The target point cloud, i.e. the sensor data recorded by the internal environmental sensor and/or the image recording device, can often overlap, in the present application, only a very small part of the source point cloud, i.e. the sensor data supplied by the external environmental sensor. However, there is a lot of sensor data in the overlap region, since the density of the target point cloud can be regularly very high. Experiments have shown that conventional solutions in the registration of the point clouds fail due to the extremely low overlap, but also due to the high degrees of freedom, i.e. normally at least a 6-DOF transformation is sought. Therefore, in this optional solution, it is suggested to use geometric boundary conditions in a first step to reduce the degrees of freedom (and optionally the point clouds themselves, see further below).

For this purpose, both point clouds can first be aligned with the direction of gravity (which can be assumed to be pointing in a vertical direction). For all the sensors, this information is generally available (e.g. from inertial measurement units (IM Us)) and/or can be derived (e.g. floor detection, alignment line detection, etc.). In the present application, the origin of the coordinate system of the medical imaging device is usually defined in its base located on the floor or the base, i.e. the target point cloud can be transformed into a coordinate system aligned with gravity with little effort. The external environmental sensor, such as AR/M R/VR glasses, may be equipped with one or more IM Us, with the result that the source point cloud can also be aligned with gravity, e.g. using accelerometers. After this alignment, the rotational search space of three axes is reduced to a single axis, namely rotation about the direction of gravity. In a second step, the translational search space can also be reduced from three to two axes. With the aid of floor detection in the source point cloud, which is often carried out in A R glasses in order to place objects on the floor, the origin of the source coordinate system or the local coordinate system of the external environmental sensor can be moved to the floor of the operating room, so that the floor has the coordinates z=0. Then, the target PCL only has to be moved in the x-axis and y-axis and rotated about the z-axis in order to determine the desired transformation rule. In a third step, a conventional cross-source point cloud registration method can be used for this.

It is conceivable that, in order to determine the first transformation rule using the predetermined cross-source point cloud registration method, only those of the further environmental sensor data whose situation in the third coordinate system is in a predetermined or determinable region are taken into account.

That is to say, apart from reducing the degrees of freedom, the source point cloud itself can also be reduced. To do this, the mean height of the target PCL above the floor can be detected or determined, with the result that the source point cloud can be cut with a buffer, thereby removing all points of the source point cloud above and below the determined height.

It is conceivable that, in order to determine the first transformation rule using the predetermined cross-source point cloud registration method, only those of the further environmental sensor data which, according to a result of a semantic segmentation carried out based on the further environmental sensor data, are assigned to a predetermined class are taken into account.

That is to say, a semantic quantity reduction can also be used to remove points from the source point cloud that potentially cannot be used for registration. For example, point cloud segmentation and classification methods can be used to assign a label to each point in the source point cloud, such as wall, floor, operating theater staff, tools, ceiling, medical imaging device, etc. All points that are not useful for alignment (e.g. ceiling, operating theater personnel, . . . ) can then be deleted in order to further reduce the size of the source point cloud.

The data processing device may be configured to recognize objects based on the environmental sensor data from the internal environmental sensor, the stereoscopic image data from the image recording device and/or the environmental sensor data from the external environmental sensor for creating the three-dimensional environmental model, and to optionally assign each of the recognized objects to one of a plurality of predetermined classes. The data processing device may be configured to determine a respective position and/or orientation of the recognized objects based on the environmental sensor data from the internal environmental sensor, the stereoscopic image data from the image recording device and/or the environmental sensor data from the external environmental sensor for creating the three-dimensional environmental model.

The data processing device can thus be configured, in order to create the three-dimensional environmental model, to recognize an object of a predetermined class based on the sensor data and to determine a position and/or orientation thereof based on the received sensor data.

That is to say, when generating the 3D model, it is possible to recognize objects that are in the environment of the medical imaging device. The recognized objects can be assigned to predetermined classes. Examples of such (object) classes are patient, medical personnel, medical apparatus and/or tool, operating theater cover (so-called drape) and/or disposable items. Optionally, the environmental model can also represent regions, objects, structures and/or surfaces (e.g. for collision avoidance) that cannot be assigned to any of these classes. In addition to the pose (position and rotation) in the global coordinate system and the form (e.g. keypoints, geometric form primitives, bounding box, mesh, and/or point cloud), an object can optionally comprise additional properties (e.g. the state of disposable items (used/unused)). Optionally, relationships between the objects will be modeled (e.g. surgeon holding forceps).

Depending on the sensor data provided, the objects can be recognized based on semantic segmentation of point clouds (i.e. in 3D) and/or based on visual imaging (i.e. in 2D).

The determination of the first transformation rule may comprise identifying one of the recognized objects contained both in the stereoscopic image data and/or the environmental sensor data and in the further environmental sensor data. The determination of the first transformation rule may comprise determining a second transformation rule, by means of which coordinates of the first coordinate system can be converted into coordinates of a third coordinate system, the coordinates of which indicate a position of a point in the environment of the medical imaging device relative to the identified object, and/or vice versa. The determination of the first transformation rule may comprise determining a third transformation rule, by means of which coordinates of the second coordinate system can be converted into coordinates of the third coordinate system, and/or vice versa. The first transformation rule can be determined based on the second and third transformation rules.

The data processing device may be configured to control operation of the medical imaging device based on the generated three-dimensional environmental model. Additionally or alternatively, the data processing device may be configured to control operation of a further (optionally medical) device based on the generated three-dimensional environmental model.

It is conceivable that a change in the actual configuration of the image recording device and/or the stand is proposed and/or this is brought about automatically (e.g., the image recording device can be centered over the situs and/or its zoom can be adjusted and/or lighting can be switched on). Additionally or alternatively, information can be sent to a user interface of the human-machine interface in order to display a representation of the generated 3D environmental model and/or to supplement displayed medical images. It is conceivable that the changes will be made automatically only when a user approves them via the user interface. The user interface optionally has different output modalities such as a screen, a touchscreen, a projector, an eyepiece (or an eyepiece for each eye) and/or a loudspeaker.

Additionally or alternatively, HM Ds can be used to display such information. Additionally or alternatively, the human-machine interface can be equipped with a microphone in order to enable a multimodal interaction concept. It is conceivable that, additionally or alternatively, gesture control of the device is possible via the internal and/or external environmental sensor.

The data processing device may be configured to control the operation of the medical imaging device in order to determine a target configuration of the medical imaging device. The data processing device may be configured to control the operation of the medical imaging device in order to automatically adapt an actual configuration of the medical imaging device to the determined target configuration. Additionally or alternatively, the data processing device may be configured to control the operation of the medical imaging device in order to output the determined target configuration to an operator of the medical imaging device by means of a human-machine interface. The human-machine interface can be part of the medical imaging device and/or can be connectable thereto.

It is conceivable that, for this purpose, predetermined properties are derived or determined from the positions and/or orientations of the recognized objects contained or modeled in the environmental model. Based on the determined properties, the target configuration of the medical imaging device can be determined. Optionally, specifications from the operator of the medical imaging device can also be taken into account. The target configuration can then be compared with the actual configuration in a target-actual comparison. The actual configuration can be actively adapted to the target configuration by controlling and/or regulating a state of the medical device. It is also conceivable that a graphic warning, a signal tone and/or a vibration is/are output depending on a result of the target-actual comparison. This can be affected via the human-machine interface. A monitor installed on the medical imaging device and/or an external monitor, e.g. a so-called head-mounted display, can be used for this purpose. It is also conceivable that information is loaded into the optical or digital eyepiece and/or a warning lamp is activated.

The predetermined (object) class can be a patient class. The data processing device may be configured to determine the position and/or orientation of an object recognized in the patient class relative to the medical imaging device. The data processing device may be configured, based on the object recognized in the patient class as the target configuration of the medical imaging device, to determine a target position and/or target orientation of the medical imaging device relative to the object recognized in the patient class and/or a target configuration of the image recording device. The data processing device may be configured to automatically adapt an actual position and/or an actual orientation of the medical imaging device to the determined target position and/or target orientation and/or to automatically adapt an actual configuration of the image recording device to the determined target configuration of the image recording device. Additionally or alternatively, the data processing device may be configured to output the determined target position and/or target orientation and/or target configuration of the image recording device to the operator of the medical imaging device by means of the human-machine interface.

It is conceivable that the patient class in turn has a subclass, which is a surgical entry point (i.e. situs) on the patient. However, the patient class can also itself be defined as the entry point, with the result that the patient is not recognized first and then the entry point, but rather the entry point is recognized directly, without the need to recognize the patient himself. It is possible to determine the position and orientation of the recognized entry point in relation to the medical imaging device. If a motorized stand (see above) is provided, a movement routine (optionally a rotation routine) of the environmental sensors mounted on the stand can be performed both for detecting the entry point and for determining its position and orientation. As described above, sensor data from further environmental sensors can also be used to provide assistance. The medical imaging system can be adjusted automatically based on the determined position and orientation of an entry point. The position and orientation of the optical system or the image recording device can be adjusted automatically, provided that a motorized stand is provided (see above). Additionally or alternatively, the magnification and/or focus of the image recording device can be adjusted based on the determined position and orientation of the entry point. It is conceivable that the system uses the human-machine interface to output an indication that it has found the entry point. In addition, it is conceivable that the human-machine interface communicates the possibility of the microscope being able to at least partially automatically align itself with the entry point. It is conceivable that the user can accept or instruct this by means of an input via the human-machine interface and the alignment is thus approved and initiated.

Optionally, the state of the 3D environmental model is (optionally continuously) recorded and stored in a memory module. Automated reports or other derivations can also be created and stored.

Optionally, the data processing device may be configured to move the internal environmental sensor to different locations and thus increase the region captured by the internal environmental sensor. Moving the internal environmental sensor makes it possible to fill any gaps in the 3D environmental model.

Optionally, the sensors themselves can be equipped with motors that allow them to be moved, optionally translationally moved and/or rotated. This can offer the advantage that such sensors can rotate independently of the robotic stand in order to change their field of view.

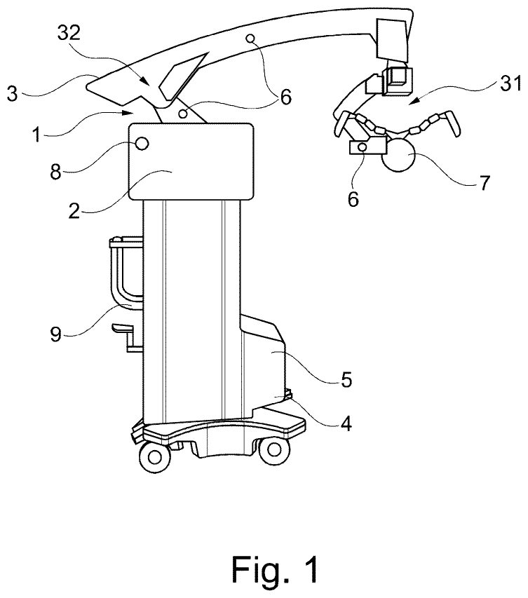

The medical imaging device 1 (referred to as a surgical microscope below) shown in FIG. 1 comprises a movable, here rollable, base 2 and a stand 3.

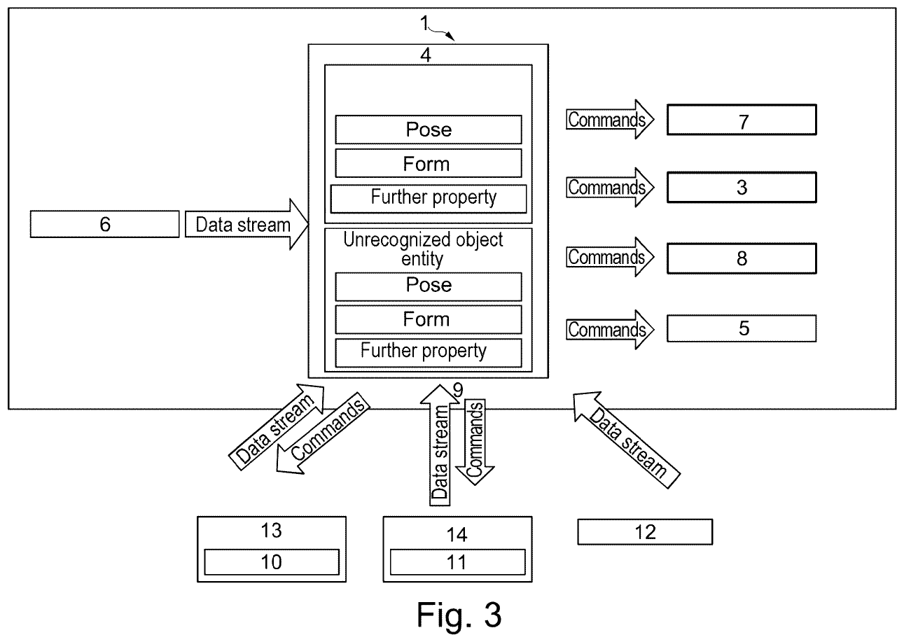

The motorized stand 3 is movably, here rotatably, hinged to the base 2. Arranged in or on the base 2 is a data processing device 4 having a memory device 5 (see also FIG. 3).

An image recording device 7 for stereoscopic image recording (referred to as a microscope below) is mounted on one end 31 (referred to as the microscope head below) of the stand 3 opposite that end 32 of the stand 3 which is hinged to the base 2. It is conceivable that the end 32 is deflected at the base 2 via a strut (not shown) which extends from the end 32 into the interior of the base 2.

A user interface 8 is also provided on the base 2 and can be used by a user to make a user input.

The data processing device 4 is connected to the user interface 8 and receives the user input, translates it into a control command for an actuator system (not shown) of the motorized stand 3 and/or the microscope head 31 and/or the microscope 7 and thus controls a movement of the stand 3 and the microscope head 31 and/or an actual configuration of the microscope 7.

Furthermore, the surgical microscope 1 comprises a plurality of, here three, environmental sensors 6. One of the environmental sensors 6 is mounted on the microscope head 31. Another of the environmental sensors 6 is mounted on the motorized stand 3 and another of the environmental sensors 6 is in turn mounted on the base 2. These environmental sensors 6 are part of the surgical microscope 1 and are therefore referred to as internal environmental sensors 6. The data processing device 4 is connected to each of the internal environmental sensors and receives environmental sensor data captured by the internal environmental sensors 6 during operation of the surgical microscope 1.

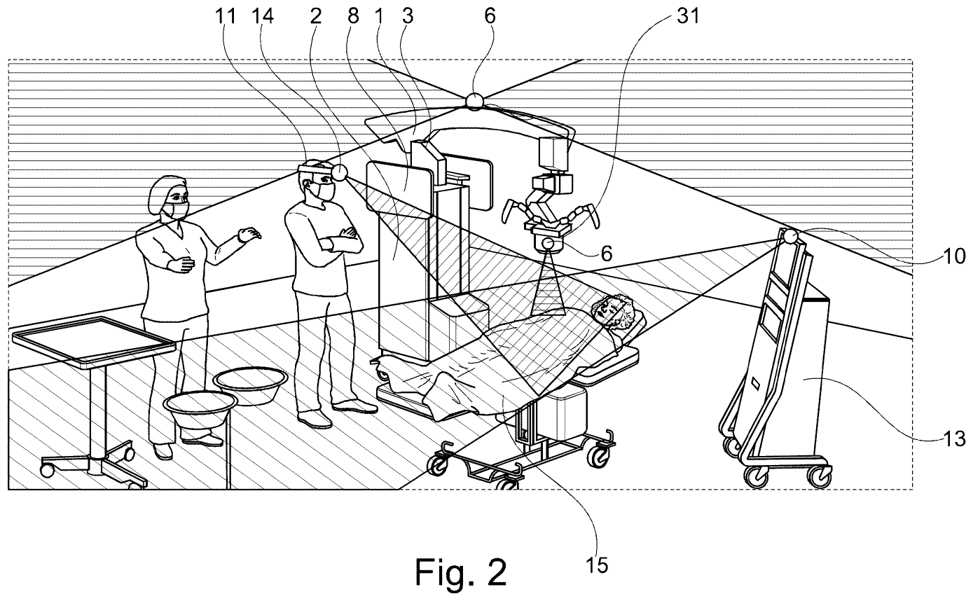

Furthermore, the surgical microscope 1 has a (network) interface 9, via which the data processing device 4 is connectable to external environmental sensors 10, 11 (see FIGS. 2 and 3).

FIG. 2 shows, by way of example, a scene from an operating room in which, in addition to the surgical microscope 1, there is a further medical device 13 which is equipped with an RGB-D camera as the first external environmental sensor 10.

Also located in the operating room is a person with a head-mounted device 14, comprising an RGB and depth camera (and a combination of an accelerometer and a gyroscope) as the second external environmental sensor 11.

The operating room may also contain a 360° RGB stereo camera (not shown) which is mounted on a ceiling of the operating room and acts as the third external environmental sensor.

As is clear from FIG. 2, the respective fields of view of the environmental sensors 6, 10, 11 overlap. For reasons of clarity, only two of the internal environmental sensors 6 are shown in FIG. 2, which are the internal environmental sensor 6 attached to the microscope head 31 and the internal environmental sensor 6 attached to the stand 3. The internal environmental sensor 6 attached to the microscope head 31 is designed as an infrared camera in the present case.

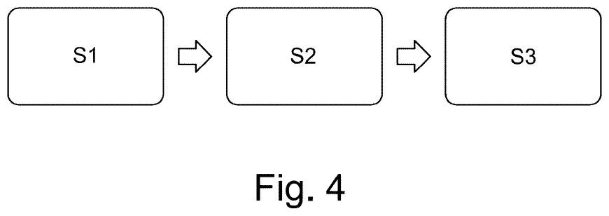

The determination according to the disclosure of a three-dimensional environmental model of the environment of the surgical microscope 1 is described below with reference to FIG. 3 which shows the data streams and control command streams between the units described above. Reference is also made to FIG. 4 which shows a flowchart of a method according to the disclosure for determining this environmental model.

In a first step S1 of the method, the data processing device 4, which is connected to the internal environmental sensors 6 (see above), receives the environmental sensor data captured by the internal environmental sensors 6. Likewise, the data processing device 4 receives the environmental sensor data from the external environmental sensors 10, 11 via the interface 9. In addition, the data processing device 4 receives the image data recorded by the microscope 7.

In a second step S2 of the method, the data processing device 4 fuses all the environmental sensor data received in the first step S1 as well as the image data recorded by the microscope 7 and determines a three-dimensional environmental model of the environment of the surgical microscope 1 based on the fused environmental sensor data. When creating the environmental model, objects of predetermined classes are recognized in the sensor data received in the first step S1 and fused, a shape of the recognized objects is optionally determined (depending on the recognized predetermined class), and a position and orientation of the recognized objects are determined. This information is stored in the environmental model. For example, one of the predetermined classes can be a patient class.

The data processing device 4 then determines the position and orientation of an object recognized in the patient class, i.e., for example, of the patient 15 shown in FIG. 2, relative to the surgical microscope 1 and stores the position and orientation in the environmental model.

Based on the three-dimensional environmental model generated, the data processing device 4 controls operation of the surgical microscope 1 in a third step S3 of the method and, via the interface 9, controls the operation of the further medical device 13 and the head-mounted device 14.

To control the surgical microscope 1, a target configuration of the surgical microscope 1 is determined based on the environmental model. In the present case, this is a target position and target orientation of the microscope 1 relative to the patient 15 and a target configuration of the microscope 1 itself

In the context of a target-actual comparison, the data processing device 4 determines a difference between the actual position and actual orientation and the target position and target orientation of the microscope 1 relative to the patient 15 and a difference between the actual configuration and the target configuration of the microscope 1.

Based on the result of the target-actual comparison, the data processing device 4 determines a recommended action or planned control of the microscope 1, the stand 3 and/or the microscope head 31 and outputs this to the medical personnel in the operating room via the user interface 8 and the head-mounted device 14.

As soon as the medical personnel has confirmed the planned control of the microscope 1, the stand 3 and/or the microscope head 31 via the user interface 8 and/or via a corresponding gesture in the context of gesture control, the microscope 1, the stand 3 and/or the microscope head 31 is/are controlled by the data processing device 4, such that the respective target configuration corresponds to the actual configuration.

The respective data streams and control commands as well as the generated environmental model are stored by the data processing device 4 for documentation purposes in the memory 5.

Sensor data fusion takes place in the second step S2 of the method. In order for this to be able to succeed, it is necessary for a geometric relationship of the respective sensors 6, 7, 10, 11 and/or the sensor data recorded by these sensors 6, 7, 10, 11 to be known. This geometric relationship is determined in the present case in the form of one or more so-called transformation rules. A transformation rule is used for coordinate transformation. In a coordinate transformation, the coordinates that a point has in one coordinate system are used to calculate the coordinates that it has in another coordinate system. From a formal point of view, this is the conversion (transformation) of the original coordinates (x1, x2, . . . , xn) into the new coordinates (x1′, x2′, . . . , xn′). Coordinate transformations are created by rotation, scaling (changing the scale), shearing and shifting (translation) of the coordinate system, which can also be combined.

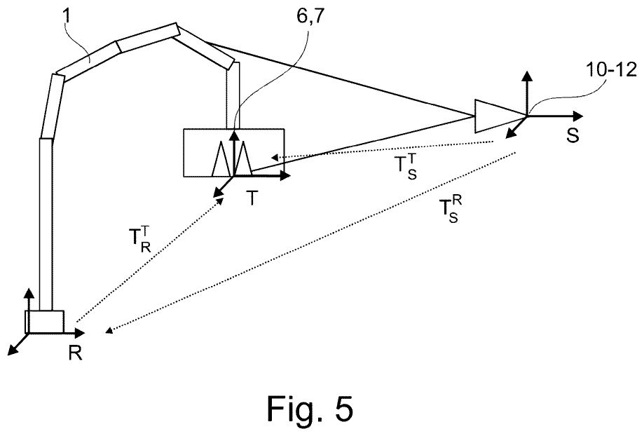

The determination of the transformation rules that is carried out in the second step S2 of the method is further explained in detail below with reference to FIGS. 5-8 which relate to a first exemplary embodiment for determining the transformation rules.

FIG. 5 shows a schematic diagram in which a spatial relationship of the medical imaging device 1, the microscope 7, the internal environmental sensor 6 and the external environmental sensor 10, 11 is shown.

As is clear from FIG. 5, a first coordinate system T is defined for the microscope 7 and/or the internal environmental sensor 6, the coordinates of which indicate a position of a point in the environment of the medical imaging device 1 relative to the microscope 7 and/or the internal environmental sensor 6. A second coordinate system S is defined for the external environmental sensor 10, 11, the coordinates of which indicate a position of a point in the environment of the medical imaging device 1 relative to the external environmental sensor 10, 11. A third coordinate system R is defined for the medical imaging device 1, the coordinates of which indicate a position of a point in the environment of the medical imaging device 1 relative to the medical imaging device 1. The coordinate systems S, T, R are each Cartesian coordinate systems.

The external environmental sensor 10, 11 is arranged and configured such that the medical imaging device 1 is at least partially arranged in its field of view and is represented by a point cloud in the environmental sensor data from the external environmental sensor 10, 11.

The fusion of the further environmental sensor data with the stereoscopic image data from the microscope 7 and/or the environmental sensor data from the internal environmental sensor 6, as carried out in the second step S2, comprises, in all exemplary embodiments, first determining a first transformation rule

$${T}_{S}^{T} ,$$

by means of which coordinates of the second coordinate system S can be converted into coordinates of the first coordinate system T, and/or vice versa.

According to the first exemplary embodiment, a second transformation rule

$${T}_{R}^{T}$$

is determined for this purpose, by means of which coordinates of the first coordinate system T can be converted into coordinates of the third coordinate system R, and/or vice versa. According to the first exemplary embodiment, a third transformation rule

$${T}_{S}^{R}$$

is also determined for this purpose, by means of which coordinates of the second coordinate system S can be converted into coordinates of the third coordinate system R, and/or vice versa. The first transformation rule

$${T}_{S}^{T}$$

is then determined based on the second and third transformation rules

$${T}_{R}^{T} , {T}_{S}^{R} .$$

The second transformation rule

$${T}_{R}^{T}$$

is determined based on the current position and orientation of the microscope 7 and/or the internal environmental sensor 6 in space and may already be available as information in the medical imaging device.

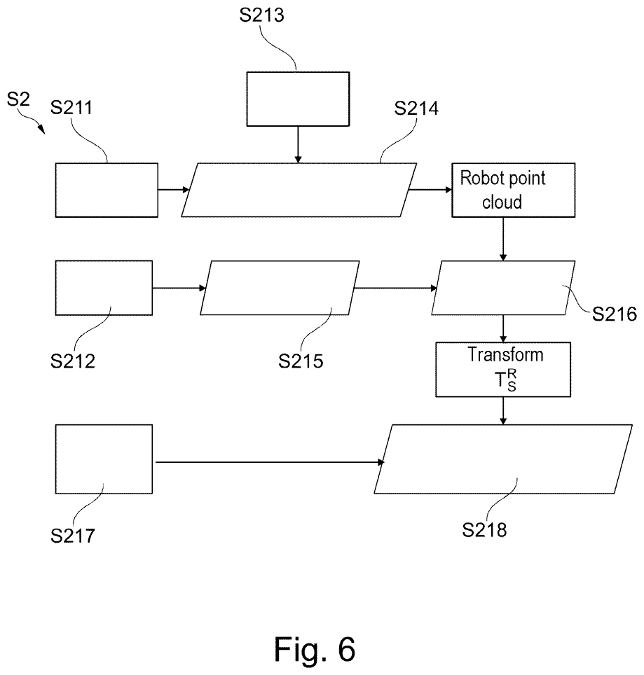

FIG. 6 shows a flowchart of a first implementation option or a first (computer-implemented) method of the first exemplary embodiment for determining the first transformation rule

$${T}_{S}^{T} ,$$

which is carried out by the data processing device 4.

In a first step S211 of the first method of the first exemplary embodiment, a 3D image (e.g. a 3D CAD model) of the medical imaging device 1 is created based on the current position and/or orientation of the image recording device 7 and/or the internal environmental sensor 6 in space. The current position and/or orientation of the image recording device 7 and/or the internal environmental sensor 6 may be available as information in the third coordinate system R, with the result that the 3D image is created in the third coordinate system R.

Furthermore, in a second step S212 of the first method, further environmental sensor data from the environmental sensor 10, 11 are provided, in which the medical imaging device 1 is represented as a first point cloud.

In a third step S213 of the first method of the first exemplary embodiment, the density of the first point cloud is provided, with the result that, in a fourth step 214 of the first method of the first exemplary embodiment, a second point cloud is derived based on the 3D image of the medical imaging device 1 and has the same density as the point cloud representing the medical imaging device 1 in the further environmental sensor data.

In a fifth step S205 of the first method of the first exemplary embodiment, a first point cloud, in which points representing the medical imaging device 1 are contained as described above, but in which other or further objects are also represented by points, is processed by means of a semantic segmentation. In this case, precisely those points which, according to the semantic segmentation, cannot be assigned to the medical imaging device 1 are filtered out.

In a sixth step S216 of the first method of the first exemplary embodiment, the second point cloud obtained in the fourth step 214 is compared with or matched to the first point cloud obtained in the fifth step 215 in order to determine the third transformation rule

$${T}_{S}^{R} .$$

This means that a 3D-3D registration takes place.

In a seventh step S217 of the first method of the first exemplary embodiment, the second transformation rule

$${T}_{R}^{T}$$

is provided, and so the first transformation rule

$${T}_{S}^{T}$$

is determined or obtained in an eighth step S218 of the first method of the first exemplary embodiment by multiplying the two aforementioned transformation rules

$${T}_{R}^{T} , {T}_{S}^{R} .$$

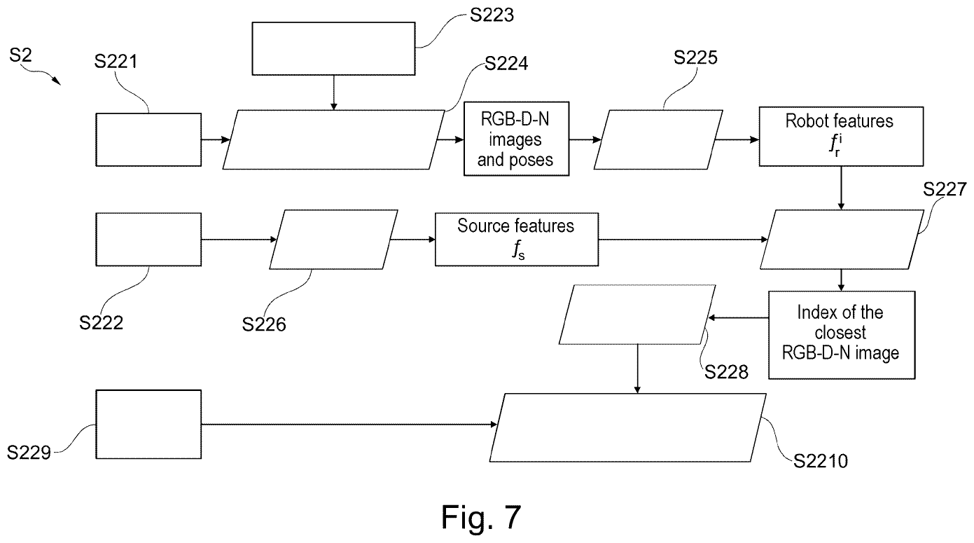

FIG. 7 shows a flowchart of a second implementation option or a second (computer-implemented) method of the first exemplary embodiment for determining the first transformation rule

$${T}_{S}^{T} ,$$

which is carried out by the data processing device 4.

In a first step S221 of the second method of the first exemplary embodiment, a 3D image of the medical imaging device 1 is obtained. This may have been determined as described above.

In a second step S222 of the second method of the first exemplary embodiment, the further environmental sensor data from the environmental sensor 10, 11, in which the medical imaging device 1 is represented as a first 2D image, are received.

In a third step S223 of the second method of the first exemplary embodiment, conditions, which may concern, for example, a height, a viewing angle of the 3D image, etc., are received, and so, in a fourth step S224 of the second method of the first exemplary embodiment, a plurality of second 2D images of the medical imaging device 1 are derived from its 3D image, as seen from different positions in the third coordinate system R, under consideration of the conditions. This is done in order to determine a position and/or orientation of the external environmental sensor 10, 11 in the third coordinate system R, from which the first 2D image of the medical imaging device 1 was recorded. The second 2D images are each a set of 2D images, each comprising an RGB image, a depth map, and a normal map.

In a fifth step S225 of the second method of the first exemplary embodiment, features, i.e. a predetermined set of features, are extracted from the second 2D images. These can be 2D features and/or image gradients.

In a sixth step S226 of the second method of the first exemplary embodiment, the same features, i.e. the predetermined set of features, are extracted from the first 2D image.

A seventh step S227 of the second method of the first exemplary embodiment comprises selecting one of the second 2D images based on a comparison of the extracted sets of features of the second 2D images with the extracted set of features of the first 2D image (so-called matching of features).

In an eighth step S228 of the second method of the first exemplary embodiment, the position in the third coordinate system R, from which the selected second 2D image was derived, is adopted as the position of the external environmental sensor 10, 11 in the third coordinate system R, from which the first 2D image of the medical imaging device 1 was recorded. The third transformation rule

$${T}_{S}^{R}$$

is then determined in this eighth step S228 based on the determined position of the external environmental sensor 10, 11 in the third coordinate system R, from which the first 2D image of the medical imaging device 1 was recorded. Depending on the type of extracted features, this can take place, for example, using a 2D-3D PnP method or algorithm, a deep learning algorithm and/or using contour-based refinement or the like.

In a ninth step S229 of the second method of the first exemplary embodiment, the second transformation rule

$${T}_{R}^{T}$$

is provided, and so the first transformation rule

$${T}_{S}^{T}$$

is determined or obtained in a tenth step S2210 of the second method of the first exemplary embodiment by multiplying the two aforementioned transformation rules.

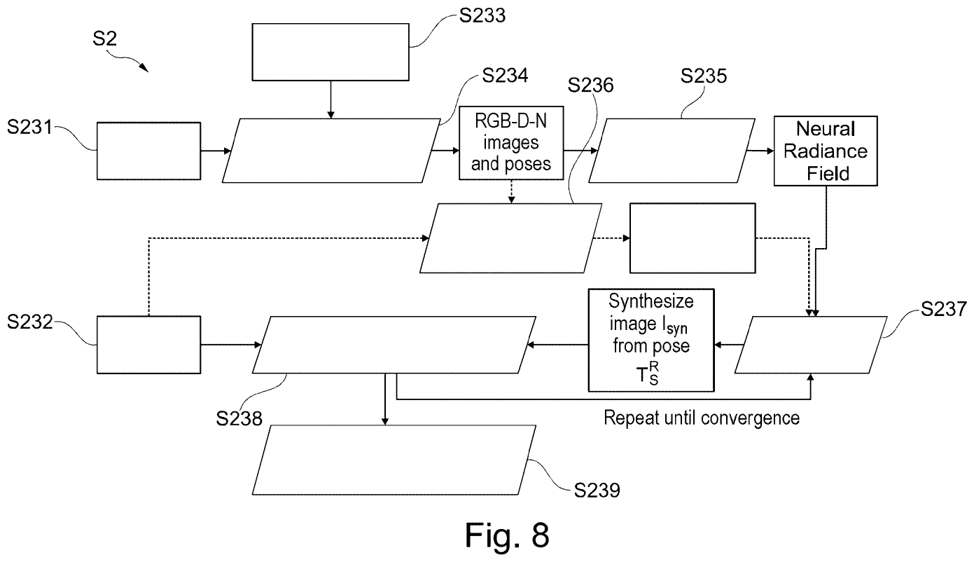

FIG. 8 shows a flowchart of a third implementation option or a third (computer-implemented) method of the first exemplary embodiment for determining the first transformation rule

$${T}_{R}^{T} , {T}_{S}^{R} .$$

which is carried out by the data processing device 4.

$${T}_{S}^{T} ,$$

In a first step S231 of the third method of the first exemplary embodiment, a 3D image of the medical imaging device 1 is obtained. This may have been determined as described above.

In a second step S232 of the third method of the first exemplary embodiment, the further environmental sensor data from the environmental sensor 10, 11, in which the medical imaging device 1 is represented as a first 2D image, are received.

In a fourth step S234 of the third method of the first exemplary embodiment, a plurality of the second 2D images of the medical imaging device 1 are derived from its 3D image, as seen from different positions in the third coordinate system R. This is done under consideration of conditions which are received in a third step S223 of the third method of the first exemplary embodiment and may concern, for example, a height, a viewing angle of the 3D image, etc. The second 2D images are each a set of 2D images, each comprising an RGB image, a depth map, and a normal map.

In a fifth step S235 of the third method of the first exemplary embodiment, an implicit scene representation is determined based on the plurality of second 2D images and the respective position in the third coordinate system R, from which the respective second 2D image was derived.

In the present case, the implicit scene representation is a neural radiance field (NeRF) model. This is an option based on deep learning for reconstructing a three-dimensional representation of a scene from a few two-dimensional images. The NeRF model makes it possible to learn a novel view synthesis, the geometry of the scene, and the reflection properties of the scene. Additional scene properties such as camera positions can also be learnt together. The NeRF makes it possible to present views from new or further viewing angles.

In a sixth step S236 of the third method of the first exemplary embodiment, a similarity search is carried out in order to determine that one of the second 2D images derived in the fourth step S234 of this method which comes closest to the first 2D image obtained in the second step S232 of this method. It is conceivable that, for this purpose, this first 2D image is compared with all (synthetically rendered) second 2D images, e.g. using a so-called place recognition method. In other words, the image similarity can be determined. The pose of this second 2D image is used to generate a further 2D image of the medical imaging device 1 based on it (see sixth step S236 of the third method). This allows the search space to be restricted or an approximate solution for an optimizer to be offered (see sixth and seventh steps S236, S237 of the third method). In other words, the pose of the synthetic image that is most similar to the first 2D image is passed to the optimizer. This allows the optimizer to converge more quickly.

In a seventh step S237 of the third method of the first exemplary embodiment, a further 2D image of the medical imaging device 1 is generated by means of the determined or generated NeRF model using a transformation rule

$$( {T}_{S}^{′ R} )$$

or the pose obtained in the sixth step S236, by means of which coordinates from the local coordinate system of the external environmental sensor 10, 11 can be converted into coordinates of the local coordinate system of the medical imaging device 1 (and/or vice versa).

In an eighth step S238 of the third method of the first exemplary embodiment, an image reconstruction loss is determined based on the further 2D image and the first 2D image.

The seventh and eighth steps S237, S238 of the third method of the first exemplary embodiment are repeated or carried out iteratively until the image reconstruction loss determined based on the last generated further 2D image and the first 2D image falls below a predetermined limit value.

In a ninth step S239 of the third method of the first exemplary embodiment, the transformation rule

$${T}_{S}^{′ R} ,$$

according to which the last generated further 2D image was generated, is adopted as the third transformation rule

$${T}_{S}^{R} .$$

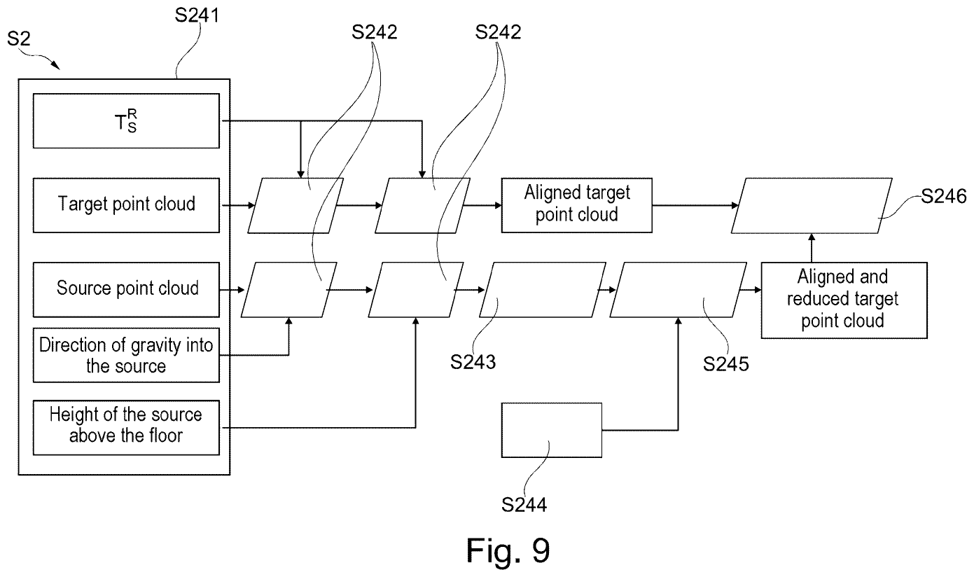

The determination of the transformation rules that is carried out in the second step S2 of the method is further explained in detail below with reference to FIGS. 5 and 9 which relate to a second exemplary embodiment for determining the transformation rules.

FIG. 9 shows a flowchart of a (computer-implemented) method of the second exemplary embodiment for determining the first transformation rule

$${T}_{S}^{T} ,$$

which is carried out by the data processing device 4.

Accordingly, the determination of the first transformation rule

$${T}_{S}^{T}$$

in a second step S242 of the method of the second exemplary embodiment comprises converting coordinates, which indicate a situation of the stereoscopic image data and/or the environmental sensor data in the first coordinate system T and a situation of the further environmental sensor data in the second coordinate system T, into a third coordinate system R. The vertical axis of the third coordinate system R runs parallel to the effective direction of the gravitational force and is perpendicular to a floor of the operating room bounding the area surrounding the medical imaging device 1 (see also FIG. 5). The origin of the third coordinate system R is located on the floor of the operating room. A situation of the stereoscopic image data and/or the environmental sensor data and the further environmental sensor data in the third coordinate system R is thus obtained.

For this purpose, in a first step S241 of the method of the second exemplary embodiment, a third transformation rule

$${T}_{S}^{R}$$

is provided, by means of which coordinates of the second coordinate system S can be converted into coordinates of the third coordinate system R. Furthermore, both the sensor data from the internal environmental sensor 6 and/or the microscope 7 and from the external environmental sensor 10, 11 are each provided as a point cloud. The external environmental sensor 10, 11 also provides information regarding the effective direction of the gravitational force relative to the external environmental sensor 10, 11 and/or its point cloud. The external environmental sensor 10, 11 also provides information regarding a height of the external environmental sensor 10, 11 and/or its point cloud relative to the floor.

In a third step S243 of the method of the second exemplary embodiment, points which are not located in a predetermined or determinable height range in the third coordinate system R are removed from the point cloud of the external environmental sensor 10, 11. The height range in the third coordinate system R is the region in which the point cloud of the internal environmental sensor 6 and/or the point cloud of the microscope 7 is/are arranged.

In a fifth step S245 of the method of the second exemplary embodiment, points which, according to a result of a semantic segmentation of the point cloud of the external environmental sensor 10, 11 that is provided in a fourth step S244 of the method of the second exemplary embodiment, cannot be assigned to the medical imaging device 1 (class) are removed from the point cloud of the external environmental sensor 10, 11.

In the sixth step S246 of the method of the second exemplary embodiment, the first transformation rule

$${T}_{S}^{T}$$

iS determined using a predetermined cross-source point cloud registration method based on the situation of the points of the point cloud of the stereoscopic image data and/or the environmental sensor data and the situation of the points of the point cloud of the further environmental sensor data, in each case in the third coordinate system R. Based on the third and fifth steps 243, 245 of the method of the second exemplary embodiment, only those points of the point cloud of the further environmental sensor data whose situation in the third coordinate system R is in the predetermined height range and which, according to the result of the semantic segmentation, are assigned to the medical imaging device 1 (class) will be taken into account for determining the first transformation rule

$$( {T}_{S}^{T} ) .$$

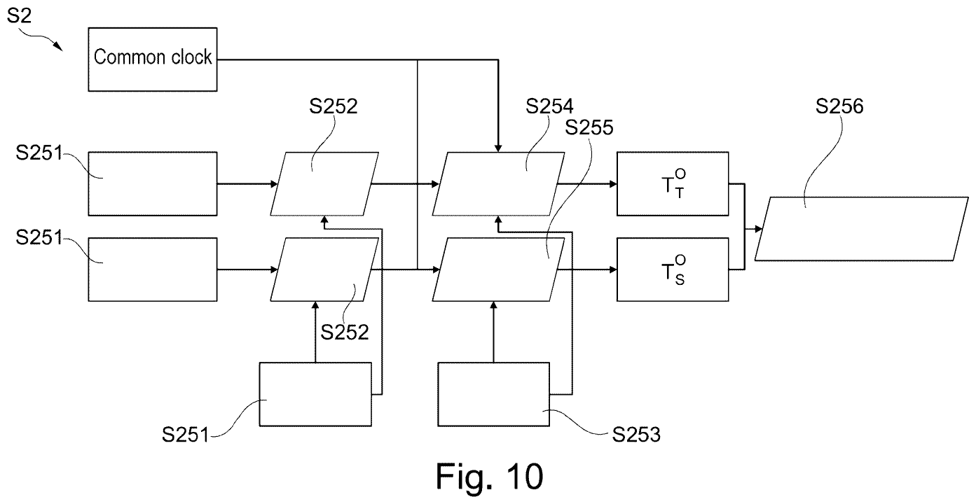

The determination of the transformation rules that is carried out in the second step S2 of the method is further explained in detail below with reference to FIGS. 5 and 10 which relate to a third exemplary embodiment for determining the transformation rules.

FIG. 10 shows a flowchart of a (computer-implemented) method of a third exemplary embodiment for determining the first transformation rule

$${T}_{S}^{T} ,$$

which is carried out by the data processing device 4.

In a first step S251 of the method of the third exemplary embodiment, environmental sensor data from the internal environmental sensor 6 and/or stereoscopic image data from the image recording device 7 as well as further environmental sensor data from the external environmental sensor 10, 11 are provided. It is conceivable that object information is also provided and allows the recognition of a predetermined object in the received sensor data.

In a second step S252 of the method of the third exemplary embodiment, one or more objects are recognized in the environmental sensor data from the internal environmental sensor 6 and/or the stereoscopic image data from the microscope 7 and the further environmental sensor data from the external environmental sensor 10, 11, in each case by means of an object recognition method. The object can be the object whose object information is provided. It is conceivable that the recognized objects are each assigned to one of a plurality of predetermined classes. One of the recognized objects, in particular the object whose object information is provided, will be identified as an object that is contained both in the stereoscopic image data and/or the environmental sensor data and in the further environmental sensor data.

In a third step S253 of the method of the third exemplary embodiment, a respective position and/or orientation of the identified object, in particular of the object whose object information is provided, is/are determined. The position and/or orientation of the identified object is/are determined in the local coordinate system T of the internal environmental sensor 6 and/or of the microscope 7 based on the environmental sensor data from the internal environmental sensor 6 and/or the stereoscopic image data from the microscope 7. The position and/or orientation of the identified object is/are also determined in the local coordinate system S of the external environmental sensor 10, 11 based on the further environmental sensor data from the external environmental sensor 10, 11.

In a fourth step S254 of the method of the third exemplary embodiment, a second transformation rule

$${T}_{T}^{O}$$

is determined by means or winch coordinates of the first coordinate system T can be converted into coordinates of a third coordinate system O, the coordinates of which indicate a position of a point in the environment of the medical imaging device 1 relative to the identified object, and/or vice versa.

In a fifth step S255 of the method of the third exemplary embodiment, a third transformation rule

$${T}_{S}^{O}$$

is determined, by means of which coordinates of the second coordinate system S can be converted into coordinates of the third coordinate system O, and/or vice versa.

In a sixth step S256 of the method of the third exemplary embodiment, the first transformation rule

$${T}_{S}^{T}$$

is determined based on the second and third transformation rules

$${T}_{T}^{O} , {T}_{S}^{O} .$$

### Clauses

C1. Computer-implemented method for creating a three-dimensional environmental model of an environment of a medical imaging device (1), characterized in that the method comprises:
   receiving sensor data from an internal sensor (6, 7) mounted on the medical imaging device (1),
   receiving further sensor data from an external environmental sensor (10, 11) arranged at a distance from the medical imaging device (1),
   determining a first transformation rule

$$( {T}_{S}^{T} ) ,$$

by means or which coordinates of a second coordinate system(S) can be converted into coordinates of a first coordinate system (T), and/or vice versa,
fusing the received sensor data with the further sensor data using the first transformation rule

$$( {T}_{S}^{T} ) ,$$

and
determining the three-dimensional environmental model based on the fused sensor data,
wherein the first coordinate system (T) is defined for the internal sensor (6, 7), the coordinates of which indicate a position of a point in the environment of the medical imaging device (1) relative to the internal sensor (6, 7), and
wherein the second coordinate system(S) is defined for the external sensor (10, 11), the coordinates of which indicate a position of a point in the environment of the medical imaging device (1) relative to the external sensor (10, 11).
C2. Method according to C1, characterized in that the determination of the first transformation rule

$$( {T}_{S}^{T} )$$

comprises:
   determining a third transformation rule

$$( {T}_{S}^{R} ) ,$$

by means or which coordinates of the second coordinate system(S) can be converted into coordinates of a third coordinate system (R), and/or vice versa, and
determining the first transformation rule

$$( {T}_{S}^{T} )$$

based on a second and the third transformation rule

$$( {T}_{R}^{T} , {T}_{S}^{R} ) .$$

wherein the third coordinate system (R) is defined for the medical imaging device (1), the coordinates of which indicate a position of a point in the environment of the medical imaging device (1) relative to the medical imaging device (1), and
wherein the second transformation rule

$$( {T}_{R}^{T} )$$

makes it possible to convert coordinates of the first coordinate system (T) into coordinates of the third coordinate system (R), and/or vice versa.
C3 Method according to C2, characterized in that the method comprises determining the second transformation rule

$$( {T}_{R}^{T} )$$

based on a current position and/or orientation of the internal sensor (6, 7) in space.
C4 Method according to C2 or C3, characterized in that the medical imaging device (1) is represented as a first point cloud in the further sensor data and the determination of the third transformation rule

$$( {T}_{S}^{R} )$$

comprises:
   creating a 3D image of the medical imaging device (1) based on a current position and/or orientation of the internal sensor (6, 7) in space,
   deriving a second point cloud based on the 3D image of the medical imaging device (1), which optionally has the same density as the first point cloud, and
   determining the third transformation rule

$$( {T}_{S}^{R} )$$

by comparing the first point cloud with the second point cloud.
C5. Method according to C2 or C3, characterized in that the medical imaging device (1) is represented as a first 2D image in the further sensor data and the determination of the third transformation rule

$$( {T}_{S}^{R} )$$

comprises:
   obtaining a 3D image of the medical imaging device (1),
   deriving at least one second 2D image of the medical imaging device (1) from its 3D image in order to determine a position of the external sensor (10, 11) in the third coordinate system (R), from which the first 2D image of the medical imaging device (1) was recorded, and
   determining the third transformation rule

$$( {T}_{S}^{R} )$$

based on the determined position of the external sensor (10, 11) in the third coordinate system (R), from which the first 2D image of the medical imaging device (1) was recorded.
C6 Method according to C5, characterized in that the determination of the position of the external sensor (10, 11) in the third coordinate system (R), from which the first 2D image of the medical imaging device (1) was recorded, comprises:
   deriving a plurality of the second 2D images of the medical imaging device (1) from its 3D image, as seen from different positions in the third coordinate system (R),
   extracting a predetermined set of features, optionally comprising 2D features and/or image gradients, from each of the first and second 2D images,
   selecting one of the second 2D images based on a comparison of the extracted sets of features of the second 2D images with the extracted set of features of the first 2D image, and
   adopting the position in the third coordinate system (R), from which the selected second 2D image was derived, as the position of the external sensor (10, 11) in the third coordinate system (R), from which the first 2D image of the medical imaging device (1) was recorded.
C7. Method according to C2 or C3, characterized in that the medical imaging device (1) is represented as a first 2D image in the further sensor data and the determination of the third transformation rule

$$( {T}_{S}^{R} )$$

comprises.
   obtaining a 3D image of the medical imaging device (1),
   deriving a plurality of the second 2D images of the medical imaging device (1) from its 3D image, as seen from different positions in the third coordinate system (R),
   determining an implicit scene representation based on the plurality of second 2D images and the respective position in the third coordinate system (R), from which the respective second 2D image was derived,
   generating further 2D images of the medical imaging device (1) by means of the determined implicit scene representation using various transformation rules

$$( {T}_{S}^{′ R} ) ,$$

wherein the further 2D image are generated iteratively until an image reconstruction loss determined based on a last generated further 2D image and the first 2D image falls below a predetermined limit value, and
adopting the transformation rule

$$( {T}_{S}^{′ R} ) ,$$

according to which the last generated further 2D image was generated, as the third transformation rule

$$( {T}_{S}^{R} ) .$$

C8 Method according to C1, characterized in that the determination of the first transformation rule

$$( {T}_{S}^{T} )$$

comprises:
   converting coordinates, which indicate a situation of the sensor data in the first coordinate system (T) and a situation of the further sensor data in the second coordinate system(S), into a third coordinate system (R), the vertical axis of which runs parallel to the effective direction of the gravitational force, in order to obtain a situation of the sensor data and the further sensor data in the third coordinate system (R), and
   determining the first transformation rule

$$( {T}_{S}^{T} )$$

using a predetermined cross-source point cloud registration method based on the situation of the sensor data and the further sensor data in the third coordinate system (R).
C9 Method according to C8, characterized in that, in order to determine the first transformation rule

$$( {T}_{S}^{T} )$$

using the predetermined cross-source point cloud registration method, only those of the further sensor data whose situation in the third coordinate system (R) is in a predetermined region are taken into account.
C10. Method according to C8 or C9, characterized in that, in order to determine the first transformation rule

$$( {T}_{S}^{T} )$$

using the predetermined cross-source point cloud registration method, only those of the further sensor data which, according to a result of a semantic segmentation carried out based on the further sensor data, are assigned to a predetermined class are taken into account.
C11. Method according to one of C1 to C10, characterized in that the method comprises:
   recognizing objects arranged in the environment of the medical imaging device based on the sensor data and the further sensor data,
   optionally, respectively assigning the recognized objects to one of a plurality of predetermined classes,
   determining a respective position and/or orientation of the recognized objects, and
   creating the three-dimensional environmental model based on the recognized objects and their respective position and/or orientation.
C12. Method according to C11, characterized in that the determination of the first transformation rule

$$( {T}_{S}^{T} )$$

identifying one of the recognized objects contained both in sensor data and in the further sensor data,
determining a second transformation rule

$$( {T}_{T}^{O} ) ,$$

by means of which coordinates of the first coordinate system (T) can be converted into coordinates of a third coordinate system (O), the coordinates of which indicate a position of a point in the environment of the medical imaging device (1) relative to the identified object, and/or vice versa, and
determining a third transformation rule

$$( {T}_{S}^{O} ) ,$$

by means of which coordinates of the second coordinate system(S) can be converted into coordinates of the third coordinate system (O), and/or vice versa,
wherein the first transformation rule

$$( {T}_{S}^{T} )$$

is determined based on the second and third transformation rules

$$( {T}_{T}^{O} , {T}_{S}^{O} ) .$$

C13. Method according to one of C1 to C12, characterized in that the method comprises controlling operation of the medical imaging device (1) based on the generated three-dimensional environmental model.
C14. Method according to C13, characterized in that the control of the operation of the medical imaging device (1) comprises:
   determining a target configuration of the medical imaging device (1), and
   automatically adapting an actual configuration of the medical imaging device (1) to the determined target configuration and/or outputting the determined target configuration to an operator of the medical imaging device (1) by means of a human-machine interface (8, 14) that is part of the medical imaging device (1) and/or is connectable thereto.
C15. Method according to C14, insofar as referred back to C11, characterized in that the predetermined class is a patient (15) class and the method comprises:
   determining the position and/or orientation of an object recognized in the patient (15) class relative to the medical imaging device (1),
   determining, as the target configuration of the medical imaging device (1), a target position and/or target orientation of the medical imaging device (1) relative to the object recognized in the patient (15) class and/or a target configuration of the internal sensor (6, 7) based on the object recognized in the patient (15) class, and
   automatically adapting an actual position and/or an actual orientation of the medical imaging device (1) to the determined target position and/or target orientation and/or an actual configuration of the internal sensor (6, 7) to the determined target configuration of the internal sensor (6, 7) and/or outputting the determined target position and/or target orientation and/or target configuration of the internal sensor (6, 7) to the operator of the medical imaging device (1) by means of the human-machine interface (8, 14).
C16. Data processing device (4), characterized in that the data processing device (4) is configured to carry out the method according to one of C1-C15.
C17. Medical imaging device (1), characterized in that the medical imaging device (1) comprises:
   an internal sensor, optionally comprising an image recording device (7) for stereoscopic image recording and/or an internal environmental sensor (6), wherein the internal sensor is mounted on the medical imaging device (1) and is configured to capture (environmental) sensor data relating to an environment of the medical imaging device (1), and
   a data processing device (4) according to C16, wherein the data processing device (4) comprises an interface which is configured to receive further (environmental) sensor data relating to the environment of the medical imaging device (1) from an external (environmental) sensor.
C18. Computer program comprising instructions which, when the computer program is executed by a computer, cause the computer to carry out the method according to one of C1-C15.
C19. Computer-readable (storage) medium comprising instructions which, when the instructions are executed by a computer, cause the computer to carry out the method according to one of C1-C15.

### LIST OF REFERENCE SIGNS

1 Medical imaging device
2 Base
3 Stand
31 First end of the stand
32 Second end of the stand
4 Data processing device
5 Memory
6 Internal environmental sensor
7 Image recording device
8 User interface
9 (Network) interface
10, 11 External environmental sensors
13 Further medical device
14 Head-mounted device
15 Patient
S1-S3 Method steps

What is claimed is:

1. A medical imaging device, comprising:
an image recording device for stereoscopic image recording,
an internal environmental sensor which is mounted on the medical imaging device and is configured to capture environmental sensor data relating to an environment of the medical imaging device, and
a data processing device which is connected to the internal environmental sensor and is configured to receive the environmental sensor data captured by the internal environmental sensor and to create a three-dimensional environmental model of the environment of the medical imaging device based on the received environmental sensor data.

2. The medical imaging device according to claim 1, wherein the data processing device is configured to be connected to the image recording device to receive stereoscopic image data from the image recording device, and to fuse the received stereoscopic image data with the environmental sensor data received from the internal environmental sensor, thereby generating the three-dimensional environmental model.

3. The medical imaging device according to claim 1, wherein the data processing device is configured:
to be connected to an external environmental sensor being arranged outside the medical imaging device and to receive further environmental sensor data from the external environmental sensor, and
to fuse the received further environmental sensor data with the environmental sensor data received from the internal environmental sensor and/or the stereoscopic image data received from the image recording device, thereby generating the three-dimensional environmental model.

4. The medical imaging device according to claim 3, wherein:
a first coordinate system is defined for the image recording device and/or the internal environmental sensor, the coordinates of which indicate a position of a point in the environment of the medical imaging device relative to the image recording device and/or the internal environmental sensor,
a second coordinate system is defined for the external environmental sensor, the coordinates of which indicate a position of a point in the environment of the medical imaging device relative to the external environmental sensor, and
the fusion of the further environmental sensor data with the stereoscopic image data and/or the environmental sensor data includes determining a first transformation rule for conversion of coordinates of the second coordinate system into coordinates of the first coordinate system, and/or vice versa.

5. The medical imaging device according to claim 4, wherein:
a third coordinate system is defined for the medical imaging device, the coordinates of which indicate a position of a point in the environment of the medical imaging device relative to the medical imaging device,
a second transformation rule is determined, the second transformation rule allowing for conversion of the coordinates of the first coordinate system into coordinates of the third coordinate system, and/or vice versa, and
the determination of the first transformation rule includes:
determining a third transformation rule allowing for conversion of the coordinates of the second coordinate system into the coordinates of the third coordinate system, and/or vice versa, and
determining the first transformation rule based on the second and third transformation rules.

6. The medical imaging device according to claim 5, wherein the data processing device is configured to determine the second transformation rule based on a current position and/or orientation of the image recording device and/or the internal environmental sensor in space.

7. The medical imaging device according to claim 5, wherein:
the medical imaging device is represented as a first point cloud in the further environmental sensor data, and
the determination of the third transformation rule includes:
creating a 3D image of the medical imaging device based on a current position and/or orientation of the image recording device and/or the internal environmental sensor in space,
deriving a second point cloud based on the 3D image of the medical imaging device, and
determining the third transformation rule by comparing the first point cloud with the second point cloud.

8. The medical imaging device according to claim 7, wherein the first and the second point cloud have a same density.

9. The medical imaging device according to claim 5, wherein:
the medical imaging device is represented as a first 2D image in the further environmental sensor data, and
the determination of the third transformation rule includes:
obtaining a 3D image of the medical imaging device,
deriving at least one second 2D image of the medical imaging device from its 3D image in order to determine a position of the external environmental sensor in the third coordinate system, from which the first 2D image of the medical imaging device was recorded, and
determining the third transformation rule based on the determined position of the external environmental sensor in the third coordinate system, from which the first 2D image of the medical imaging device was recorded.

10. The medical imaging device according to claim 9, wherein the determination of the position of the external environmental sensor in the third coordinate system, from which the first 2D image of the medical imaging device was recorded, includes:
deriving a plurality of the second 2D images of the medical imaging device from its 3D image, as seen from different positions in the third coordinate system,
extracting a predetermined set of features from each of the first and second 2D images,
selecting one of the second 2D images based on a comparison of the extracted sets of features of the second 2D images with the extracted set of features of the first 2D image, and
adopting the position in the third coordinate system, from which the selected second 2D image was derived, as the position of the external environmental sensor in the third coordinate system, from which the first 2D image of the medical imaging device was recorded.

11. The medical imaging device according to claim 10, wherein the predetermined set of features comprises 2D features and/or image gradients.

12. The medical imaging device according to claim 5, wherein:
the medical imaging device is represented as a first 2D image in the further environmental sensor data, and
the determination of the third transformation rule comprises:
obtaining a 3D image of the medical imaging device,
deriving a plurality of second 2D images of the medical imaging device from its 3D image, as seen from different positions in the third coordinate system,
determining an implicit scene representation based on the plurality of the second 2D images and the respective position in the third coordinate system, from which a respective second 2D image was derived,
generating further 2D images of the medical imaging device based on the determined implicit scene representation using further transformation rules, wherein the further 2D images are generated iteratively until an image reconstruction loss determined based on a last generated further 2D image and the first 2D image falls below a predetermined limit value, and
adopting a transformation rule of the further transformation rules, according to which a last one of the further 2D image was generated, as the third transformation rule.

13. The medical imaging device according to claim 4, wherein the determination of the first transformation rule includes:
converting coordinates, which indicate a situation of the stereoscopic image data and/or the environmental sensor data in the first coordinate system and a situation of the further environmental sensor data in the second coordinate system, into a third coordinate system, a vertical axis of which runs parallel to an effective direction of a gravitational force, in order to obtain a situation of the stereoscopic image data and/or the environmental sensor data and the further environmental sensor data in the third coordinate system, and
determining the first transformation rule using a predetermined cross-source point cloud registration method based on the situation of the stereoscopic image data and/or the environmental sensor data and the further environmental sensor data in the third coordinate system.

14. The medical imaging device according to claim 13, wherein, for determining the first transformation rule using the predetermined cross-source point cloud registration method, only those of the further environmental sensor data whose situation in the third coordinate system is in a predetermined region are taken into account.

15. The medical imaging device according to claim 13, wherein, for determining the first transformation rule using the predetermined cross-source point cloud registration method, only those of the further environmental sensor data which, according to a result of a semantic segmentation carried out based on the further environmental sensor data, are assigned to a predetermined class are taken into account.

16. The medical imaging device according to claim 1, wherein the data processing device (4) is configured to recognize objects and to determine a respective position and/or orientation of the recognized objects based on the environmental sensor data from the internal environmental sensor, the stereoscopic image data from the image recording device, and/or further environmental sensor data from an external environmental sensor, thereby creating the three-dimensional environmental model, wherein the data processing device is configured to be connected to the external environmental sensor being arranged outside the medical imaging device and to receive the further environmental sensor data from the external environmental sensor.

17. The medical imaging device according to claim 16, wherein:
the data processing device is configured to fuse the received further environmental sensor data with the environmental sensor data received from the internal environmental sensor and/or the stereoscopic image data received from the image recording device thereby generating the three-dimensional environmental model,
a first coordinate system is defined for the image recording device and/or the internal environmental sensor, the coordinates of which indicate a position of a point in the environment of the medical imaging device relative to the image recording device and/or the internal environmental sensor,
a second coordinate system is defined for the external environmental sensor, the coordinates of which indicate a position of a point in the environment of the medical imaging device relative to the external environmental sensor,
the fusion of the further environmental sensor data with the stereoscopic image data and/or the environmental sensor data includes determining a first transformation rule for conversion of coordinates of the second coordinate system into coordinates of the first coordinate system, and/or vice versa,
wherein the determination of a first transformation rule includes:
identifying one of the recognized objects contained both in the stereoscopic image data and/or the environmental sensor data and in the further environmental sensor data,
determining a second transformation rule allowing for conversion of the coordinates of the first coordinate into coordinates of a third coordinate system, the coordinates of which indicate a position of a point in the environment of the medical imaging device relative to the identified object, and/or vice versa, and
determining a third transformation rule allowing for conversion of the coordinates of the second coordinate system into the coordinates of the third coordinate system, and/or vice versa,
wherein the first transformation rule is determined based on the second and third transformation rules.

18. The medical imaging device according to claim 1, wherein the data processing device is configured to control an operation of the medical imaging device based on a generated three-dimensional environmental model.

19. The medical imaging device according to claim 18, wherein the data processing device, for controlling the operation of the medical imaging device, is configured:
to determine a target configuration of the medical imaging device, and
to adapt an actual configuration of the medical imaging device to the determined target configuration and/or to output the determined target configuration to an operator of the medical imaging device (via a human-machine interface that is part of the medical imaging device and/or is connectable thereto.

20. The medical imaging device according to claim 16, wherein
the data processing device is configured to assign each of the recognized objects to one of a plurality of predefined classes, wherein one of the plurality of predefined classes is a patient class and the data processing device (4) is configured:
to determine the position and/or orientation of an object recognized in the patient class relative to the medical imaging device,
based on the object recognized in the patient class as a target configuration of the medical imaging device, to determine a target position and/or target orientation of the medical imaging device relative to the object recognized in the patient class and/or a target configuration of the image recording device, and
to adapt an actual position and/or an actual orientation of the medical imaging device to the determined target position and/or target orientation and/or an actual configuration of the image recording device to the determined target configuration of the image recording device and/or to output the determined target position and/or target orientation and/or target configuration of the image recording device to an operator of the medical imaging device via a human-machine interface.
